(19) United States
(12) Patent Application Publication
(10) Pub. No.: US 2017/0231940 A1
(43) Pub. Date: Aug. 17, 2017

(54) Multiparticulate L-Carnitine And Nootropic Compositions And Related Methods

(71) Applicant: Physician's Seal, LLC, Boca Raton, FL (US)

(72) Inventors: Syed M. Shah, Boca Raton, FL (US); Noreen Hassan, Boca Raton, FL (US); Christopher Diorio, Boca Raton, FL (US); Fred Hassan, Boca Raton, FL (US)

(73) Assignee: Physician's Seal, LLC, Boca Raton, FL (US)

(21) Appl. No.: 15/586,652

(22) Filed: May 4, 2017

Related U.S. Application Data
(63) Continuation-in-part of application No. 14/476,152, filed on Sep. 3, 2014.
Application No. 14/476,152 is a continuation of application No. 13/490,198, filed on Jun. 6, 2012, now Pat. No. 8,828,426.
(60) Provisional application No. 61/494,053, filed on Jun. 7, 2011.
(60) Provisional application No. 62/332,683, filed on May 6, 2016.

(51) Int. Cl. A61K 31/205; A61K 45/06; A61K 31/198; A61K 9/16; A61K 31/7068

(52) U.S. Cl. CPC A61K 31/205; A61K 9/167; A61K 31/7068; A61K 31/198; A61K 45/06

(57) ABSTRACT

A composition includes a therapeutically effective pharmaceutical dosage form including a plurality of individual particulates. The individual particulates respectively have: a core including an active ingredient combination of an L-carnitine and a nootropic substance and a release controlling polymer over the core that substantially prevents release of the active ingredients in stomach acid and permits release of the active ingredients in an intestinal pH environment. The composition may be used to treat conditions associated with a reduction of the amount of L-carnitine in the body and/or cognitive impairment.

### CROSS-REFERENCE TO RELATED APPLICATIONS

This is a continuation-in-part of application Ser. No. 14/476,152, filed Sep. 3, 2014, which is continuation of application Ser. No. 13/490,198, now U.S. Pat. No. 8,828,426, filed Jun. 6, 2012, which claims priority to U.S. provisional Application No. 61/494,053, filed Jun. 7, 2011. This also claims priority from provisional Application No. 62/332,683, filed May 6, 2016. These prior applications are incorporated by reference herein in their entireties.

### FIELD

This relates to therapeutic compositions, and more particularly, to therapeutic compositions that provide controlled delivery of an L-carnitine and a nootropic substance.

### BACKGROUND

L-Carnitine is a naturally occurring compound that facilitates the transport of fatty acids into the mitochondria for oxidation, and thereby energy production. It is a derivative of the amino acid lysine. Acetyl L-Carnitine (“ALCAR”) is an acetylated derivative of L-Carnitine.

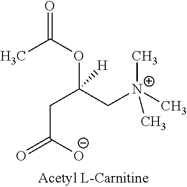

L-carnitine and its derivatives are used to treat various physiological conditions such as carnitine deficiency, decline in mitochondrial function, cognitive function, cardiovascular disease (myocardial infarction, heart failure and angina pectoris), intermittent claudication, end-stage renal failure, Alzheimer's disease, HIV/AIDS and decreased sperm motility.

In humans, the endogenous carnitine pool includes free L-carnitine and a range of short-, medium- and long-chain esterified carnitine derivatives, including ALCAR and propionyl-L carnitine, for example. As a supplement taken orally, ALCAR has been reported to have better bioavailability than L-carnitine itself. Therefore, the esterified forms of L-carnitine may be considered as prodrugs of L-carnitine.

Nootropics are a class of compounds that are reported to improve cognitive functions by modulating neurotransmitter activity. Examples of nootropics include aceglutamide, racetams, aniracetam, oxiracetam, piracetam, pramicetam, oxiracetam, ampakines, sunifiram, noopept, bifematlane, exifone, fipexide, idebenone, indeloxazune hydrochloride, nizofenone, vincamine, propentofylline, pyritinol, tacrine, vinpocetine, L-serine, and citicholine.

L-carnitine, ALCAR, and certain nootropics are commonly used dietary supplements for improving cognitive functions such as alertness, memory, and concentration. They are typically sold in single unit dosage forms such as monolithic tablets. These single unit dosage forms often do not provide a controlled release of the active ingredient.

Unfortunately, L-carnitines may cause side effects such as upset stomach, nausea and vomiting when released in the stomach. Thus, it is desirable to administer L-carnitines in a manner that minimizes the manifestation of these side effects. Current administration techniques are hampered in this regard because they present a small therapeutic window between when the benefits of L-carnitines take effect and when the side effects are manifested.

### BRIEF SUMMARY

A first example of the composition comprises a therapeutically effective pharmaceutical dosage form including a plurality of individual particulates. The individual particulates respectively have: a core including an active ingredient combination of an L-carnitine and a nootropic substance and a release controlling polymer over the core that substantially prevents release of the active ingredients in stomach acid and permits release of the active ingredients in an intestinal pH environment.

A second example of the composition comprises a therapeutically effective pharmaceutical dosage form including a plurality of individual particulates. The individual particulates respectively have by % w/w of the particulate: a solid core including 35% to 65% w/w of an L-carnitine; 1% to 20% w/w of a subcoating over the solid core; 15% to 30% w/w of a nootropic substance that is within the core or within the subcoating; and an enteric coating over the subcoating.

An example of a method of treatment for a physiological condition associated with cognitive impairment comprises administering to a patient in need thereof a therapeutically effective pharmaceutical dosage form including a plurality of individual particulates. The individual particulates respectively have a core including an active ingredient combination of an L-carnitine and a nootropic substance and a release controlling polymer over the core that substantially prevents release of the active ingredient combination in stomach acid and permits release of the active ingredient combination in an intestinal pH environment.

The following additional features may be included in any of these examples of compositions and methods.

The L-carnitine may be within an interior solid portion of the core and the nootropic substance is within a coating over the interior solid portion of the core.

The core may include a solid interior portion and the L-carnitine and nootropic substance are in the solid interior portion of the core.

The L-carnitine may be selected from at least one of L-carnitine, acetyl L-carnitine, and propionyl L-carnitine.

The nootropic substance may be selected from at least one of L-serine and citicoline.

An average diameter of the individual particulates may be 0.1 to 3 mm.

The therapeutically effective pharmaceutical dosage form may include 300 mg to 800 mg of the L-carnitine and 25 mg to 300 mg of the nootropic substance.

The therapeutically effective pharmaceutical dosage form may include 400 mg to 600 mg of the L-carnitine and 200 mg to 250 mg of the nootropic substance.

The individual particulates may respectively include by % w/w of the particulate: 35% to 65% w/w of the L-carnitine and 15% to 30% w/w of the nootropic substance.

The individual particulates may respectively include by % w/w of the particulate: 45% to 55% w/w of the L-carnitine and 20% to 30% w/w of the nootropic substance.

The dosage form may be adapted to release the L-carnitine and nootropic substance within the pH range found in the intestines continuously for at least 3 and up to 10 hours.

The dosage form may be effective to release the L-carnitine and nootropic substance over a period of 3-10 hours after ingestion.

### DETAILED DESCRIPTION OF EXAMPLE EMBODIMENTS

As used below, the term “L-carnitine” refers to L-carnitine-containing compounds such as L-carnitine and its derivatives, including acetyl L-carnitine and propionyl L-carnitine for example.

Examples of nootropics that may be used include, but are not limited to, include aceglutamide, racetams, aniracetam, oxiracetam, piracetam, pramicetam, oxiracetam, ampakines, sunifiram, noopept, bifematlane, exifone, fipexide, idebenone, indeloxazune hydrochloride, nizofenone, vincamine, propentofylline, pyritinol, tacrine, vinpocetine, L-serine, and citicholine.

The composition may be used to treat physiological conditions related to a reduction of L-carnitine in the body of a patient. Examples of physiological conditions that may be treated with such a composition include L-carnitine deficiency, decline in mitochondrial function, cardiovascular disease (myocardial infarction, heart failure and angina pectoris), intermittent claudication, end-stage renal failure, Alzheimer's disease, Parkinson's disease, Peyronie's disease, insulin response deficiencies, HIV/AIDS, peripheral nerve injury, spinal cord injury, and decreased sperm motility.

The composition may also be used to treat conditions associated with cognitive impairment such as to improve alertness, memory, and/or concentration.

The composition may provide reduced release in the stomach and an elevated release at a substantially neutral pH, such as the pH found in the intestines. As used herein, a substantially neutral pH environment means an environment having a pH of about 7, including, but not limited to a pH of between about 6.5 to about 7.5, also including the pH environment of the intestines.

In a particular example the dosage form is a multiparticulate dosage form that includes a plurality of individual particulates that can pass to the intestines without substantially releasing the L-carnitine and nootropic in the stomach, thus preventing the undesirable side effects or reduced efficacy of L-carnitine that may result otherwise.

The multiparticulate composition includes a plurality of individual particulates that may be generally spheroidal in shape and may be incorporated into a capsule or packet-type oral delivery dosage form.

The individual particulates are sized to fit through the pyloric sphincter in a relaxed state. The diameter of the particulates may be in the range of about 0.1 to about 3 mm, about 0.5 mm to about 2.5 mm, about 1 mm to about 2.5 mm, or about 1 mm to about 2 mm. The individual particulates are independently dispersible in the stomach and intestines.

The particulates include a solid core containing the L-carnitine. The nootropic substance may be within the interior portion of the solid core with the L-carnitine. Alternatively, the nootropic substance may be placed in a coating over the core.

Examples of excipients that may be used to prepare the particulates include, but are not limited to, carriers, diluents, disintegrants, emulsifiers, solvents, processing aids, buffering agents, colorants, flavorings, solvents, coating agents, binders, carriers, glidants, lubricants, granulating agents, gelling agents, polishing agents, suspending agent, sweetening agent, anti-adherents, preservatives, emulsifiers, antioxidants, plasticizers, surfactants, viscosity agents, enteric agents, wetting agents, thickening agents, stabilizing agents, solubilizing agents, bioadhesives, film forming agents, emollients, dissolution enhancers, dispersing agents, or combinations thereof.

Conventional processing aids may be used to prepare the particulates. Examples of processing aids include, but are not limited to, magnesium stearate, stearic acid, talc, and sodium lauryl sulfate.

The particulates may include a pharmaceutically acceptable filler. Examples of fillers include, but are not limited to, silicates, calcium carbonate, glycine, dextrin, sucrose, sorbitol, dicalcium phosphate, calcium sulfate, lactose, kaolin, mannitol, sodium chloride, talc, dry starches, microcrystalline cellulose, and powdered sugar.

The particulates may include a pharmaceutically acceptable binder. Examples of binders include, but are not limited to, cellulosic, and povidone binders such as hypromellose, microcrystalline cellulose, and crospovidone.

The particulates may be coated to aid in swallowing, to mask the taste of the ingredients, improve appearance, to protect the dosage form from moisture, and/or to have an enteric coating. The coating may be applied using conventional coating techniques, such as, for example, spray coating, bed coating, or the like.

The particulates may be coated with a release controlling polymer that is over the core that substantially prevents release of the active ingredient combination in stomach acid and permits release of the active ingredients in an intestinal pH environment.

An example of such a release controlling coating is an enteric coating. The enteric coating to substantially prevent the active ingredients from releasing into the stomach. Examples of enteric coating materials include shellac, cellulose acetate phthalate, polyvinyl acetate phthalate, ethyl cellulose/sodium alginate, hypromellose acetate succinate, or a methacrylic acid-based polymer or co-polymer such as methacrylic acid-ethyl acrylate copolymer.

The particulates may be coated with a seal coating. Examples of seal coating materials include, but are not limited to, hydroxypropyl cellulose, hypromellose, and polyvinyl alcohol. A particular example of the seal coating is OPADRY Clear, which contains, HPMC and polyethylene glycol.

A subcoating may be placed over the core between the core and the release controlling coating or enteric coating. The subcoat is effective to prevent the materials making up the release controlling coating or enteric coating from reacting with or otherwise interfering with the core. It may also prevent intrusion of water and air into the core during storage. The subcoating may be made from a binder solution applied over the core. The solution may be, for example, an aqueous 10% hypromellose solution.

The multiparticulate composition provides an advantageous non-parenteral delivery vehicle that can be administered to a patient.

The individual particulates may be combined to form a unitary multiparticulate dosage form such as by being combined in a tablet, caplet, capsule, sachet, or the like. A particular example of the dosage form is capsule or sachet containing the individual particulates.

Several particular examples of the composition will now be described. The scope of possible embodiments, however, is not limited to these examples. These examples are presented as percent by weight (% w/w) of the specified ingredient relative to the dosage form. If an enteric coating is placed over the dosage form, the % w/w is the pre-enteric or release controlling coating % w/w. Any combination of the ingredients in the % w/w listed below may be employed.

The L-carnitine may be 20%-80% w/w; 30%-80% w/w, 35%-75% w/w, 35%-65% w/w, 40%-60% w/w, 35%-55% w/w, 45%-55% w/w, or 48%-52% w/w.

The nootropic substance may be 1%-40% w/w; 2%-30% w/w; 2.5%-30% w/w; 10%-40% w/w; 15%-30% w/w; 20%-30% w/w; or 20%-25% w/w.

The filler may be 5%-60% w/w; 10%-50% w/w; 5%-25% w/w; 10%-20% w/w; 10%-25% w/w; 12%-20% w/w; or 13%-19% w/w.

The binder may be 1%-40% w/w; 5%-30% w/w; 5%-15% w/w, 7%-17% w/w; 8% -11% w/w; 4%-6% w/w.

The subcoat may be 1%-20% w/w; 1.5%-15% w/w; 2%-10% w/w; 1%-5% w/w; or 2%-4% w/w.

The enteric coating or release control coating may be 5%-30% w/w; 5%-25% w/w; or 8%-25% w/w.

In terms of weight gain, the enteric coating or release controlling coating may be applied such that the weight of the subcoated core increases by 10%-30% w/w due to the added coating.

The nootropic substance may be within the core such that it is blended with the other materials forming the core. In such an example, the core may include:
20%-80% w/w, 30%-80% w/w, 35%-75% w/w, 35%-65% w/w, 40%-60% w/w, 35%-55% w/w, 45%-55% w/w, or 48%-52% w/w of the L-carnitine;
1%-40% w/w, 2%-30% w/w, 2.5%-30% w/w, 10%-40% w/w, 15%-30% w/w, 20%-30% w/w, or 20%-25% w/w of the nootropic substance;
5%-60% w/w, 10%-50% w/w, 5%-25% w/w, 10%-20% w/w, 10%-25% w/w, 12%-20% w/w, or 13%-19% w/w filler; and
1%-40% w/w, 5%-30% w/w, 5%-15% w/w, 7%-17% w/w, 8%-11% w/w, or 4%-6% w/w binder.

In another example, the nootropic substance may be applied over the core as part of a coating. In such an example, the core may include:
20%-80% w/w, 30%-80% w/w, 35%-75% w/w, 35%-65% w/w, 40%-60% w/w, 35%-55% w/w, 45%-55% w/w, or 48%-52% w/w of the L-carnitine;
5%-60% w/w, 10%-50% w/w, 5%-25% w/w, 10%-20% w/w, 10%-25% w/w, 12%-20% w/w, or 13%-19% w/w filler; and
1%-40% w/w, 5%-30% w/w, 5%-15% w/w, 7%-17% w/w, 8%-11% w/w, or 4%-6% w/w binder.
   The core is coated with a coating including the nootropic substance such that the coated core is 1%-40% w/w, 2%-30% w/w, 2.5%-30% w/w, 10%-40% w/w, 15%-30% w/w, 20%-30% w/w, or 20%-25% w/w of the nootropic substance.

In another example, the nootropic substance is blended with the material forming the subcoating such that the nootropic substance becomes part of the subcoating. In such an example, the nootropic substance is blended with the coating material and a solvent such that the nootropic substance and coating material are intermixed. When the subcoating is applied to the core and dried, the nootropic substance is dispersed in the subcoating.

It should be understood that the filler, binder, subcoating, release controlling coating and/or enteric coating may not be necessary for every dosage form.

A therapeutically effective amount is an amount that is sufficient to affect a disease or process in the body. A particular example dose of a multiparticulate composition may provide about 1000 mg to 3000 mg or, more preferably, about 2000 mg of L-carnitine. Doses of the multiparticulate composition may be administered sporadically. A patient may be a human or animal patient.

A therapeutically effective amount of the L-carnitine and/or nootropic ingredient may be 1-10,000 mg/day, including 1-25 mg/day, 25-50 mg/day, 50-75 mg/day, 75-100 mg/day, 100-150 mg/day, 150-200 mg/day, 200-250 mg/day, 250-300 mg/day, 300-350 mg/day, 350-400 mg/day, 400-450 mg/day, 450-500 mg/day, 500-550 mg/day, 550-600 mg/day, 600-650 mg/day, 650-700 mg/day, 700-750 mg/day, 750-800 mg/day, 800-850 mg/day, 850-900 mg/day, 900-950 mg/day, 950-1,000 mg/day. Higher doses (1,000-6,000 mg/day) might also be effective. The weight in mg is often calibrated to the body weight of the patient in kg, thus these example doses may also be written in terms of mg/kg of body weight per day.

In practice, the therapeutically effective amount may vary depending on numerous factors associated with the patient, including age, weight, height, severity of the condition, administration technique, and other factors. The therapeutically effective amount administered to a patient may be determined by medical personnel taking into account the relevant circumstances.

The therapeutically effective amount may be determined or predicted from empirical evidence. Specific dosages may vary according to numerous factors and may be initially determined on the basis of experimentation.

The product may be administered as a single dose or as part of a dosage regimen. For a dosage regimen, the therapeutically effective amount is adjustable dose to dose to provide a desired therapeutic response.

Multiple doses may be administered at a predetermined time interval and subsequent doses may be proportionally reduced or increased, depending on the situation.

Table 1 is a list of a few of the possible therapeutically effective amounts of active ingredients in several examples of the composition, Examples A-H. Here, the mass is reported by weight of a plurality of particulates forming a unitary dosage form. This list is not meant to be exhaustive.

TABLE 1
Amounts of active ingredients in examples of the composition

| | Mass (milligrams) | | | | | | | |
|---|---|---|---|---|---|---|---|---|
| Active | A | B | C | D | E | F | G | H |
| The L- | 300-800 | 400-700 | 400-600 | 450-550 | 300-700 | 350-650 | 475-525 | 480-520 |
| carnitine | | | | | | | | |
| Nootropic | 25-300 | 50-300 | 200-300 | 150-350 | 150-250 | 200-250 | 205-245 | 215-235 |
| substance | | | | | | | | |

Methods of making the multiparticulate compositions will now be described. The may be prepared by wet granulating the core materials into a wet mass, extruding the wet mass to form an extrudate, cutting the extrudate into a plurality of core pieces, and spheronizing the core pieces. The spheronized core pieces are may be dried to <3% based on the Karl Fischer method.

The spheronized core pieces are then coated with the subcoating and dried before being coated with the enteric coating or release control coating material, which are typically applied in a fluidized bed coater. The coated particulates may subsequently be dried, to <3% (Karl Fischer).

The dried coated multiparticulates may then be prepared into a suitable pharmaceutical dosage form such as a capsule, sachet, or tablet, for example. A typical preferred capsule contains about 1000 mg of the particulates. Depending on the desired dosage, however, this may be adjusted.

The multiparticulate composition may be formulated to be taken orally by a human or animal patient and to ensure that the patient receives an effective amount of L-carnitine and nootropic substance over the course of several hours after ingestion.

The composition may be used to treat at least one of carnitine deficiency, age related decline in mitochondrial function, cardiovascular disease (myocardial infarction, heart failure and angina pectoris), intermittent claudication, end-stage renal failure, Alzheimer's disease, HIV/AIDS and decreased sperm motility. The method of treatment includes administering an effective amount of the composition to a patient in need thereof.

The term “patient” refers to humans or other animals considered as having one or more physiological conditions that can be remediated with the active ingredients.

The term “administering” refers to the giving or applying of a substance. In one example embodiment, administering the composition to the patient includes orally administering an oral dosage form to the patient. The oral dosage form may be a capsule having the independently dispersible particulates therein, for example.

Administering the composition to the patient may include combining the independently dispersible particulates with an acidic food vehicle, such as an acidic, semi-solid food or drink. This administration technique may be particularly useful with patients who have difficulty swallowing. The particulates may be loaded into a sachet that the patient or a caregiver can easily open for sprinkling the particulates onto the acidic food vehicle. When the patient ingests the acidic food vehicle, the patient also ingests the particulates. Examples of acidic food vehicles include food products like applesauce, fruit slurries, fruit juices, or the like.

The independently dispersible particulates may be administered to a patient using a feeding tube such as a gastric feeding tube, nasogastric feeding tube, or jejunostomy feeding tube.

Doses of the multiparticulate composition may be administered sporadically when needed or may be administered as part of a long term treatment.

The composition has many advantages. Some but not all of those advantages are listed here. Not all of the advantages are required by all embodiments of the composition.

The composition may provide a more reliable release of the L-carnitine and nootropic substance when compared to single-unit sustained release formulations that are presently available, without concern for dosing of the patient under the fed or fasted state.

It may also provide a prolonged exposure to the L-carnitine and nootropic substance locally and systemically as compared to the single-unit sustained release formulations.

The composition may allow for less frequent dosing and may also allow for dosing with a lower total amount of L-carnitine and nootropic. Dispersion of the particulates in the lumen of the small bowel, prior to release of the L-carnitine and nootropic substance, may reduce the incidence of side effects seen with other L-carnitine formulations.

The independently dispersible particulates will disperse in the intestines tract to provide a delocalized dose of the L-carnitine and nootropic substance therein.

### EXAMPLES

This section describes a few specific examples of the composition. These examples are presented by way of example only and are not intended to limit the scope of the possible embodiments.

### Example 1

Table 2 lists ingredients in an example of the core. Table 3 lists ingredients in an example of enteric coated particulates.

TABLE 2
Ingredients of an exemplary embodiment of the core.

| | (grams/ | Ingredient |
|---|---|---|
| Ingredient | % w/w) | Function |
| L-Carnitine | 935.9/78.4 | Active ingredient |
| CAB-O-SIL M5P | 19.1/1.6 | Processing Aid |
| (Colloidal Silicon | | |
| Dioxide) | | |
| AVICEL (Micro | 227.0/19.0 | Filler |
| Crystalline cellulose) | | |
| Hypromellose | 11.9/1.0 | Binder |
| (METHOCEL ® A15 | | |
| Premium) | | |
| Water (% of | (15.0%) | |
| dry mass) | | |

TABLE 3
Ingredients of an exemplary embodiment
of enteric-coated particulates.

| | (grams/ | Ingredient |
|---|---|---|
| Ingredient | % w/w) | Function |
| KOLLICOAT ® | 506.6/85.8 | Source of |
| MAE 30 DP Solids | | methacrylic |
| | | copolymer |
| Triethyl Citrate | 75.7/12.8 | Plasticizer |
| PLASACRYL ® T20 | 7.9/1.3 | Anti-Adherent |
| Water¹ | | |

¹Evaporates

### Preparation of Multiparticulate Compositions

Experimental Details. The equipment that may be utilized to create the compositions herein include the following: top loading balances, hand screens (12, 14, 16, 18, Pan, 70 mesh), Rotap sieve shaker, IKA mixer, KitchenAid food processor (pre-milling), Hobart mixer, LCI Benchtop Granulator, Fitz mill equipped with a 0.065″ screen, Jet Mill, Key International high sheer mixer, Glatt GPCC-3 fluid bed drier, Glatt GPCC-3 fluid bed dried with 7″ Wurster, Karl Fischer moisture analyzer, and a spheronizer.

Acetyl L-Carnitine pre-conditioning. The Acetyl-L-Carnitine raw material contains large clumps of fine crystals. Also, the material is hygroscopic. It is necessary to de-lump the raw material and reduce the hygroscopicity in order to process the material. 500 g Acetyl-L-Carnitine (Lonza ALC Carnipure) and 10.2 grams CAB-O-SIL M5P (Cabot Corporation) were blended for 1-5 minutes in a KitchenAid Food Processor equipped with blade or similar blender and equipped with intensifier bar or pin bar.

Preparation of Core. The core was prepared utilizing the following steps and settings. 955 grams ALCAR/SiO₂ (98/2% w/w), 227 grams Microcrystalline Cellulose (Avicel Ph 102; FMC Corporation), and 11.9 grams Methocel A15 LV (Dow) were low shear granulated in a 0.5 Gallon (2 Liter) Hobart or other granulation mixer and mixed at low speed for about 5 minutes. About 162-172 g USP water was sprayed into the mixer to achieve peak granulation moisture of about 12% to about 12.6% w/w, and this was blended for about an additional 10-30 minutes to form a wet mass.

The wet mass was extruded through a 1.0 mm-hole perforated metal screen using a LCI Benchtop Granulator at speed setting 10.

The extrudate was spheronized in 25-30 grams sub lots using a Caleva Model 120 spheronizer equipped with a small pyramid plate at high speed for 2-3 minutes.

The combined spheronization sub lots (˜1373 grams) were dried in a GPCG-3 or similar fluid bed dryer for about 45 minutes with an inlet temperature set point of 50° C. and a process air flow of 60 cfm.

The finished dried ALCAR multiparticulates were collected between 12-mesh and 18-mesh screens resulting in a loose Bulk Density of about 0.68 g/cc. A Camsizer particle size distribution analysis was performed finding a size distribution of: DV₁₀ 1.002 mm, DV₅₀ 1.177 mm, and DV₉₀ 1.405 mm; a specific surface area (Sv) of 5.132/mm; and a specific surface area (Sm) of 75.923 cm²/g.

Application of sub-coating. 1000 grams of ALCAR particulates were separated based on their size. The fraction that fell within the 14-18 mesh size were chosen for sub-coating. The cores were placed into a Glatt GPCC-3 fluid bed drier and the sub-coating was sprayed onto the cores in the form of a 10% hypromellose (hypromellose E5) aqueous solution that was at room temperature.

The sub-coating solution (306 g USP Water (T>55° C.) and 34 g hypromellose E5) was applied to the cores using the following parameters: the inlet temperature was maintained at about 50° C.; the air flow was maintained at about 50 cfm; the spray rate was maintained between 6.0 and 11.0 g/min; and the filter shake cycle was 45/3 seconds (Time Between Shaking/Shaking Time). The fluid bed drier was setup with a 1.0 mm Schlick 970 nozzle port, and 2X360 air cap setting, a 1.5 cm partition setting, and a multiparticulate bottom plate or equivalent.

Preparation of enteric coating solutions. The enteric coatings were applied to the cores in a fluidized bed coater (7″ wurster) as a liquid solution. The formula for the enteric coating was 1160 grams USP Water (RT), 506.6 grams BASF KOLLICOAT MAE 30 DP, 75.7 grams PLASACRYL® T20 (Colorcon), and 7.9 grams triethyl citrate USP, which was mixed a minimum of 20 minutes and screen through a 40-mesh screen prior to use.

The enteric coating solution was applied to 1000 grams of ALCAR particulate cores using the following parameters: the inlet temperature was maintained at about 50° C.; the air flow was maintained at about 50 cfm; the spray rate was maintained between 6.0 and 11.0 g/min; the atomization air pressure was maintained at about 2.0 bar; and the filter shake cycle was 45/3 seconds (Time Between Shaking/Shaking Time). The fluid bed drier was set up with a 1.0 mm Schlick 970 nozzle port, and 2X360 air cap setting, a 1.5 cm partition setting, and a multiparticulate bottom plate or equivalent.

A finish coat may be applied over the enteric coating, and is applied in a same or similar manner as the enteric coating.

### Example 2

In this prospective example, the L-carnitine and nootropic substance are within the core. The range represents additional examples.

The dosage form is a multiparticulate and the total mass represents the total mass of the individual particulates in the dosage form.

TABLE 4
Contents of example dosage form

| | | | Range |
|---|---|---|---|
| | Ingredient | mg | (mg) |
| Core | L-carnitine or acetyl L-carnitine | 500 | 300-800 |
| | or propionyl L-carnitine | | |
| | microcrystalline cellulose | 150 | 100-500 |
| | Hypromellose | 100 | 50-300 |
| | (METHOCEL ® K4M) | | |
| Sub | hypromellose | 25 | 15-100 |
| coating | (PHARMACOAT ® 615) | | |
| | citicholine or L-serine | 225 | 25-300 |
| | Total mass | 1000 | |

| | | |
|---|---|---|
| Enteric | KOLLICOAT ® MAE 30, | 10-30% |
| Coating | triethyl citrate, PLASACRYL ® | Weight gain |

### Example 3

In this prospective example, the nootropic substance is within the core. The range represents additional examples.

The dosage form is a multiparticulate and the total mass represents the total mass of the individual particulates in the dosage form.

TABLE 5
Contents of example dosage form

| | | mg/ | Range |
|---|---|---|---|
| | Ingredient | gram | (mg) |
| Core | L-carnitine or acetyl L-carnitine | 500 | 300-800 |
| | or propionyl L-carnitine | | |
| | citicholine or L-serine | 250 | 25-300 |
| | microcrystalline cellulose | 175 | 100-500 |
| | Hypromellose | | |
| | (METHOCEL ® K4M) | 50 | 50-300 |
| Sub | hypromellose | | |
| coating | (PHARMACOAT ® 615) | 25 | 15-100 |
| | Total mass | 1000 | |

| | | |
|---|---|---|
| Enteric | Kollicoat MAE 30, | 10-30% |
| Coating | triethyl citrate, Plasacryl | Weight gain |

This disclosure has described example embodiments, but not all possible embodiments of the composition or associated methods. Where a particular feature is disclosed in the context of a particular embodiment, that feature can also be used, to the extent possible, in combination with and/or in the context of other embodiments. The composition and related methods may be embodied in many different forms and should not be construed as limited to only the embodiments described here. This disclosure describes exemplary embodiments, but not all possible embodiments of the composition and methods. Where a particular feature is disclosed in the context of a particular embodiment, that feature can also be used, to the extent possible, in combination with and/or in the context of other embodiments.

That which is claimed is:

1. A composition comprising a therapeutically effective pharmaceutical dosage form including a plurality of individual particulates, the individual particulates respectively having:
a core including an active ingredient combination of an L-carnitine and a nootropic substance; and
a release controlling polymer over the core that substantially prevents release of the active ingredient combination in stomach acid and permits release of the active ingredient combination an intestinal pH environment.

2. The composition of claim 1, wherein the L-carnitine is within an interior solid portion of the core and the nootropic substance is within a coating over the interior solid portion of the core.

3. The composition of claim 1, wherein the core includes a solid interior portion and the L-carnitine and nootropic substance are in the solid interior portion of the core.

4. The composition of claim 1, wherein the L-carnitine is selected from at least one of L-carnitine, acetyl L-carnitine, and propionyl L-carnitine.

5. The composition of claim 1, wherein the nootropic substance is selected from at least one of L-serine and citicoline.

6. The composition of claim 1, wherein an average diameter of the individual particulates is 0.1 to 3 mm.

7. The composition of claim 1, wherein the therapeutically effective pharmaceutical dosage form includes 300 mg to 800 mg of the L-carnitine and 25 mg to 300 mg of the nootropic substance.

8. The composition of claim 1, wherein the therapeutically effective pharmaceutical dosage form includes 400 mg to 600 mg of the L-carnitine and 200 mg to 250 mg of the nootropic substance.

9. The composition of claim 1, wherein the L-carnitine is in an amount that is at least two times greater than the nootropic substance.

10. The composition of claim 1, wherein the individual particulates respectively include by % w/w of the particulate: 35% to 65% w/w of the L-carnitine and 15% to 30% w/w of the nootropic substance.

11. The composition of claim 1, wherein the individual particulates respectively include by % w/w of the particulate: 45% to 55% w/w of the L-carnitine and 20% to 30% w/w of the nootropic substance.

12. The composition of claim 1, wherein:
the individual particulates respectively include by %w/w of the particulate: 35% to 65% w/w of the L-carnitine and 15% to 30% w/w of the nootropic substance;
the therapeutically effective pharmaceutical dosage form includes 300 mg to 800 mg of the L-carnitine and 25 mg to 300 mg of the nootropic substance;
an average diameter of the individual particulates is 0.1 to 3 mm; and
the nootropic substance is selected from at least one of L-serine and citicoline.

13. A composition comprising a therapeutically effective pharmaceutical dosage form including a plurality of individual particulates, the individual particulates respectively having by % w/w of the particulate:
a solid core including 35% to 65% w/w of an L-carnitine;
1% to 20% w/w of a subcoating over the solid core;
15% to 30% w/w of a nootropic substance that is within the solid core or within the subcoating; and
an enteric coating over the subcoating.

14. The composition of claim 13, wherein the core includes a solid interior portion and the L-carnitine and nootropic substance are in the solid interior portion of the core.

15. The composition of claim 13, wherein the L-carnitine is selected from at least one of L-carnitine, acetyl L-carnitine, and propionyl L-carnitine.

16. The composition of claim 13, wherein an average diameter of the individual particulates is 0.1 mm to 3 mm.

17. The composition of claim 13, wherein the nootropic substance is selected from at least one of L-serine and citicoline.

18. The composition of claim 13, wherein the individual particulates respectively include by % w/w of the particulate: 45% to 55% w/w of the L-carnitine and 20% to 30% w/w of the nootropic substance.

19. The composition of claim 13, wherein the L-carnitine is in an amount that is at least two times greater than the nootropic substance.

20. The composition of claim 13, wherein therapeutically effective pharmaceutical dosage form includes 300 mg to 800 mg of the L-carnitine and 25 mg to 300 mg of the nootropic substance.

21. The composition of claim 13, wherein:
an average diameter of the individual particulates is 0.1 mm to 3 mm;
the nootropic substance is selected from at least one of L-serine and citicoline;
the individual particulates respectively include by % w/w of the particulate: 45% to 55% w/w of the L-carnitine and 20% to 30% w/w of the nootropic substance; and
therapeutically effective pharmaceutical dosage form includes 300 mg to 800 mg of the L-carnitine and 25 mg to 300 mg of the nootropic substance.

22. A method of treating a physiological condition associated with cognitive impairment, the method comprising:
administering to a patient in need thereof a therapeutically effective pharmaceutical dosage form including a plurality of individual particulates, the individual particulates respectively having:
a core including an active ingredient combination of an L-carnitine and a nootropic substance; and
a release controlling polymer over the core that substantially prevents release of the active ingredient combination in stomach acid and permits release of the active ingredient combination in an intestinal pH environment.

23. The method of claim 22, wherein administering the therapeutically effective pharmaceutical dosage form to the patient comprises administering at least one capsule containing the plurality of individual particulates therein.

24. The method of claim 22, wherein the L-carnitine is within an interior solid portion of the core and the nootropic substance is within a coating over the interior solid portion of the core.

25. The method of claim 22, wherein the core includes a solid interior portion and the L-carnitine and nootropic substance are in the solid interior portion of the core.

26. The method of claim 22, wherein the L-carnitine is selected from at least one of L-carnitine, acetyl L-carnitine, and propionyl L-carnitine.

27. The method of claim 22, wherein the nootropic substance is selected from at least one of L-serine and citicoline.

28. The method of claim 22, wherein an average diameter of the individual particulates is 0.1 to 3 mm.

29. The method of claim 22, wherein the therapeutically effective pharmaceutical dosage form includes 300 mg to 800 mg of the L-carnitine and 25 mg to 300 mg of the nootropic substance.

30. The method of claim 22, wherein the therapeutically effective pharmaceutical dosage form includes 400 mg to 600 mg of the L-carnitine and 200 mg to 250 mg of the nootropic substance.

31. The method of claim 22, wherein the L-carnitine is in an amount that is at least two times greater than the nootropic substance.

32. The method of claim 22, wherein the individual particulates respectively include by % w/w of the particulate: 35% to 65% w/w of the L-carnitine and 15% to 30% w/w of the nootropic substance.

33. The method of claim 22, wherein the individual particulates respectively include by % w/w of the particulate: 45% to 55% w/w of the L-carnitine and 20% to 30% w/w of the nootropic substance.

34. The method of claim 22, wherein:
the individual particulates respectively include by % w/w of the particulate: 35% to 65% w/w of the L-carnitine and 15% to 30% w/w of the nootropic substance;
the therapeutically effective pharmaceutical dosage form includes 300 mg to 800 mg of the L-carnitine and 25 mg to 300 mg of the nootropic substance;
an average diameter of the individual particulates is 0.1 to 3 mm; and
the nootropic substance is selected from at least one of L-serine and citicoline.
